(19) United States
(12) Patent Application Publication
(10) Pub. No.: US 2011/0160677 A1
(43) Pub. Date: Jun. 30, 2011

(54) Dropper Device

(75) Inventor: Graham Alan March, Surrey (GB)

(73) Assignee: Special Products Limited, Weybridge (GB)

(21) Appl. No.: 12/974,889

(22) Filed: Dec. 21, 2010

(30) Foreign Application Priority Data
Dec. 22, 2009 (GB) 0922357.9
Jun. 18, 2010 (GB) 1010268.9

(51) Int. Cl. A61M 5/31; B65B 3/02; B65D 71/00; B29C 45/00

(52) U.S. Cl. 604/212; 53/452; 206/232; 264/328.1

(57) ABSTRACT

Unit-dose containers and kits for administering liquid medicament to the buccal cavity of a patient, and related methods of manufacturing such containers, are described. The containers may include a chamber, with a liquid medicament housed therein, and a squeezable region used to deploy the medicament. The container also includes sealing means to prevent premature deployment of the medicament. The container also includes an elongated dispensing tube, approximately 1 to 12 cm in length, in fluid communication with the chamber. The length of the dispensing tube may be sufficient to allow, for example, the distal end of the tube to be located within the buccal cavity of most patients, without the user's hand having to be located between the jaws of the patient.

The present invention relates to a container for storing and dispensing a liquid medicament. In particular, the liquid medicament is for buccal administration.

Pharmaceutical compositions may be administered to those in need thereof via a range of routes. A number of pharmaceutical compositions are administered buccally as drugs administered in this way may be absorbed rapidly through the mucous membranes of the mouth, without being exposed to acidic degradation in the stomach or to enzymatic degradation within the GI tract as for drugs administered orally.

It is believed that the first attempt to use the buccal route for drug delivery was made by Sobrero in 1847. Since that time, a considerable amount of research into this route of delivery has been undertaken (Advances in Buccal Delivery, RAJ et al., Clinical reviews in therapeutic drug carrier systems, 2005 Vol 22, Issue 3, page 125). However there are still relatively few drugs on the market that utilise this route of delivery.

A small number of liquid pharmaceutical products formulated specifically for administration via the buccal route are available. An example of such a product is Epistatus, which is a composition comprising midazolam (Special Products Limited, UK). Epistatus has been available in the UK for eight years.

As a result of the benefits provided by administration via the buccal route, there has been considerable interest in reformulating drugs which have conventionally been administered via other routes.

Additionally, a number of drugs formulated for administration via other routes, such as injectable solutions, have been used to treat patients buccally, for example morphine, midazolam and diamorphine. However, there is little documentation on their use via the buccal route.

Historically, buccally administered medicaments, whether formulated for administration via the buccal route or via other routes, have been administered using syringes. Most commonly, the buccal compositions are provided as bulk liquids. A dose of the liquid is then drawn from the bulk liquid in a syringe and administered to a patient.

By preparing doses in this way, there is a risk that an incorrect dose will be drawn from the bulk liquid as a result of error on the part of the person withdrawing the liquid from the bulk supply.

Further, a number of drugs which are administered buccally are controlled substances. As a requirement of legislation in most countries, access to bulk supplies of such drugs is carefully managed. If a patient is in urgent need of the drug, locating and accessing the bulk supply of the drug will add to the time required to calculate, obtain and administer the dose.

A further drawback is that once a syringe has been filled and is being taken to the patient, a relatively low amount of pressure on the plunger end of the syringe will result in inadvertent discharge of the medicament.

In an attempt to address these disadvantages, pre-filled syringes were introduced. These syringes, as the name suggests, are provided pre-filled with the correct dose of medicament. They are capped over the dispensing end of the syringe to prevent inadvertent discharge of the medicament.

However, disadvantages common to both conventional and pre-filled syringes remain unaddressed. More specifically, several drugs administered via the buccal route are for the treatment of uncooperative patient groups, which makes it difficult to accurately administer drugs via a syringe. For example, buccal midazolam is used to treat epileptic patients who are suffering seizures. Administering medicaments accurately to the buccal cavity of such patients is difficult, especially when the dexterity and coordination to operate a syringe is required.

Morphine is an example of a drug formulated as a solution for administration via injection which is occasionally administered to paediatric patients in hospitals via the buccal route. This provides a rapid onset of analgesia without increasing the levels of anxiety in paediatric patients which may arise through the use of an intravenous syringe. Paediatric patients with pain levels requiring the use of opiates are unlikely to remain still and be co-operative when drugs are administered buccally, especially when faced with a syringe even if no needle is present.

As mentioned above, several medicaments administered via the buccal route are controlled substances. When either a pre-filled or conventional syringe is used to deliver these substances, the possibility exists for a person having access to the syringe to covertly remove a portion of the medicament for illicit use and administer the remaining medicament to a patient.

Additionally, both conventional and pre-filled syringes are usually only used for a single administration of medicament, prior to being discarded. Despite this, the construction of syringes is relatively complex, requiring the collocation of a several components.

Accordingly, there is a long felt need for a device which can be used to administer precise doses of medicament to the buccal cavity of a patient, which can be used to rapidly administer doses of medicament to a patient, which can be used to improve the ease of administering medicament to the buccal cavity of uncooperative patient groups, which is less susceptible to inadvertent discharge and which prevents the covert removal of medicament for illicit use.

### SUMMARY OF THE INVENTION

Thus, according to a first aspect of the present invention, there is provided a unit-dose container for administering liquid medicament to the buccal cavity of a patient comprising a chamber, a squeezable region, sealing means, and a dispensing tube in fluid communication with the chamber, the dispensing tube comprising a dispensing feature and having a length of about 1 to about 12 cm.

According to a second aspect of the present invention, there is provided a kit comprising the container of the above first aspect together with instructions for administration of medicament to a patient via the buccal cavity using the container.

According to a third aspect of the present invention, there is provided a method for preparing the container of the above first aspect using injection moulding techniques.

The present invention provides a container for administration of a single dose of a liquid medicament to the buccal cavity of a patient in need thereof.

Use of the containers of the present invention eliminates the risk of incorrect doses of medicament being administered to patients. This is advantageous not only to the patient, for obvious reasons, but also to the person administering the medicament, who will not be exposed to accusations of inadvertently or intentionally overdosing (or underdosing) patients. This advantage is of particular benefit where the medicament is a controlled substance.

The containers of the present invention provide a convenient solution to a long felt need in the art. The dispensing tube allows convenient administration of the medicament to the buccal cavities of patients, without requiring the dexterity or co-ordination required to administer liquid medicaments to patients, especially uncooperative patient groups, using a syringe.

The squeezable region of the container is adapted to be squeezed, preferably manually (i.e. without the use of mechanical apparatus) to force substantially all of the liquid medicament stored in the container through the dispensing tube. It will be appreciated that while parts of the container may be compressible by the user, such parts do not constitute a squeezable region within the context of the present invention unless they result in substantially all of the dose stored within the container being dispensed therefrom.

The squeezable region of the container is typically deformed by squeezing between two or more fingers or a thumb and one or more fingers. By squeezing the container, the medicament is forced towards the dispensing tube. In preferred arrangements of the present invention, the squeezable region of the container is at least part of the container wall defining the chamber.

It is preferable that the container be formed of a transparent or at least translucent material to allow the user to clearly determine, when administering the medicament, whether the chamber and/or tube has been emptied.

The material from which the containers are formed preferably has a degree of rigidity as this allows the dispensing feature to be more easily located in the buccal cavity of a patient than if the material is highly flexible.

Further, it is preferred that the material from which the containers are manufactured be inert with respect to the active ingredient and excipients present in the medicament.

In preferred embodiments, the container is formed, at least partially, from polymeric plastics material, for example polyolefins such as polyethylene, polypropylene, polyester or mixtures thereof or other such materials which are known by those skilled in the art to be suitable for use in the manufacture of containers of the present invention.

In especially preferred embodiments, the container is provided in its entirety from the same material.

Recycling spent containers which are formed mostly, if not totally, of the same material, is less problematic than recycling syringes, which include components formed of different materials.

Typically the polymeric material has thermoplastic properties allowing the container to be formed in a mould, for example, by injection moulding. In an especially preferred arrangement, the container is formed using blow-fill-seal apparatus. When blow-fill-seal apparatus is employed, the containers may be provided singly, or as ‘cards’ of a plurality of containers.

Preparing containers in this way is a less complex process than preparing syringes which are conventionally used to administer liquid medicaments to the buccal cavity of patients.

The container walls must be of sufficient thickness to effectively house the medicament and protect it from being punctured or burst when inadvertently exposed to forces during transport and use. At the same time, the walls forming the squeezable region of the container must also be of a thickness which permits compression or squeezing to force the liquid housed in the container through the dispensing tube. In preferred embodiments, the thickness of the container wall ranges from about 0.25 to about 2.0 mm, about 0.4 to about 1.5 mm, or, most preferably, from about 0.5 to about 1.0 mm.

The chamber may take any shape in cross-section provided that, when the container is squeezed, substantially all of the medicament is dispensed via the tube. It is especially preferred that the container will have a lozenge, diamond or circular shape in cross-section. It is also preferred that, whichever shape of chamber is employed, any corners will be rounded to prevent medicament becoming trapped in those corners upon administration of the medicament from the container. In preferred embodiments, the chamber has a symmetrical profile.

It is envisaged that the container of the present invention will be capable of storing any quantity of medicament. However, as the container of the present invention is a unit dose container, i.e. one containing only a single dose of medicament, the chamber will typically be arranged to store a volume of about 0.1 to about 20 ml of medicament. In more preferred embodiments, the chamber will be adapted to store about 0.25 to about 5 ml, or more preferably, about 0.5 to about 2 ml of medicament.

In preferred embodiments of the present invention, the container of the present invention houses a volume of about 0.1 to about 20 ml of medicament. In more preferred embodiments, the container will house about 0.2 to about 5 ml, 0.25 to about 3 ml, 0.3 to about 2.4 ml or more preferably, about 0.5 to about 2 ml of medicament.

In preferred arrangements of the present invention, the container is provided with a fill line. The purpose of the fill line is to enable the user, in the event that they suspect leakage of the stored dosage, to hold the container steadily to allow the liquid to settle. The level of the settled liquid will align with the fill line if there has been no leakage.

The container may also bear information relating, for example to the name of the drug, the batch number or the expiry date of the product. This may be printed onto the container using ink, or embossed in the inner or outer surface of the container wall. Preferably, an identification tab is provided on which such information may be presented.

The sealing means must be removed before the medicament may be administered. The sealing means can take any form provided that egress of the medicament from the container is prevented prior to removal of the sealing means.

The sealing means can be situated at any location on the container. For example, the sealing means may be located on or in the tube, or it may be located in the transition region between the chamber and the tube. In a preferred embodiment, the sealing means is located at the distal end of the tube.

In addition to the sealing means needing to prevent egress of the medicament from the container, a further requirement is that it is capable of being removed with relative ease.

In a preferred arrangement, the sealing means is a frangible seal. Such a seal may be prepared by blocking the dispensing feature of the tube and forming a weakened portion of the tube in the region which is adjacent to the blocked portion of the tube. In this embodiment or in other embodiments, the frangible seal may be provided with a tab, which allows the frangible seal to be easily removed from the container. The tab enables the user to grip and apply sufficient force upon the frangible seal to remove it from the container.

In an especially preferred arrangement, the frangible seal is formed such that removal of the tab does not leave a jagged edge or burrs at the end of the tube from which it was removed. As those skilled in the art will appreciate, the formation of such a seal can be achieved using a range of manufacturing techniques. For example, the tab can be formed including a bubble, where the tube is sealed by an outer wall of the bubble. In such arrangements, a region of the tube adjacent to the bubble may be weakened to facilitate removal of the tab.

A significant advantage provided by the frangible seal is that the illicit removal of medicament from the container prior to administration of the medicament to a patient is prevented as those present when the dose is administered will be able to clearly determine whether the container has been opened for the first time immediately prior to administration.

While the frangible seal is arranged to be removable upon the application of manual force by the user, the level and direction of force required to remove the seal will be greater than those forces to which the container will be exposed when, for example, in transit or when being handled by users prior to removal of the seal. Accordingly, the risk of inadvertent discharge of medicament is minimised.

In alternative embodiments of the present invention, the sealing means may take the form of a cap placed over the dispensing feature of the tube which cap may be adhered to the tube using adhesive or may be pressure fit against the outer surface of the tube. In such arrangements a tab may also be provided on the cap to assist with its removal.

Once the sealing means is removed, the dispensing feature of the tube is positioned in the patient's buccal cavity and the container squeezed to administer the medicament.

The tube has a length of about 1 to about 12 cm. This tube length is sufficient to allow the dispensing feature of the tube to be located in the buccal cavity of the patient, while the user's hand is a safe distance from the mouth of the patient, which is especially important for certain patient groups, for example, epileptic patients undergoing seizures. In more preferred embodiments, the tube has a length of about 1.5 to about 10 cm, about 2 to about 9 cm, about 2.5 to about 8 cm, about 3 to about 7 cm, about 3.5 to about 6.5 cm or most preferably, from about 4 to about 6 cm.

It is preferred that the container be arranged such that the distance between the squeezable region and the dispensing feature be sufficient to enable the liquid to be dispensed while minimising the risk of the user being bitten by the patient. Preferably, the distance between the squeezable region and the dispensing feature is about 1 to about 12 cm. In more preferred embodiments, the distance between the squeezable region and the dispensing feature is about 1.5 to about 10 cm, about 2 to 9 cm, about 2.5 to about 8 cm, about 3 to about 7 cm, about 3.5 to about 6.5 cm or most preferably, from about 4 to about 6 cm.

Medicament is dispensed through the dispensing feature of the tube. The dispensing feature may consist of a single orifice at the distal end of the tube. In such arrangements, the orifice may be formed when the sealing means are removed.

In alternative embodiments, the dispensing feature of the tube may be an orifice provided in the side tube wall toward the distal end of the dispensing tube. Alternatively, several orifices may be provided along the side wall and optionally at the distal end of the dispensing tube to increase the area of the buccal mucosa to which medicament is administered.

The tube may take any shape in cross-section, although tubes of circular or elliptical cross section are preferred. The exterior and/or interior of the tube may be of substantially constant diameter or the diameter may vary. In preferred embodiments, the exterior and/or interior of the tube has generally constant cross-section. In alternative embodiments, the tube may have a broader exterior and/or interior cross-section at the end which contacts the chamber and a narrower cross section at the distal end of the tube. In especially preferred embodiments of the present invention, the minimum interior and/or exterior diameter of the tube is at least about 70% of the maximum interior and/or exterior diameter of the tube. In more preferred embodiments, the minimum interior and/or exterior diameter of the tube is at least about 80%, 85%, 90% or even 95% of the maximum interior and/or exterior diameter of the tube.

The tube is preferably linear or straight and is free of changes in angle or direction. Although non-linear tubes (i.e. bent or curved tubes) may be employed in the containers of the present invention, these are generally less preferable as the bend or angle in the tube makes accurately locating the dispensing feature into the buccal cavity of the patient challenging.

In further embodiments, the tube may be provided with formations such as bulbs, constrictions, annular flanges, etc. However, such projections from the surface of the tube are generally less preferable as they may interfere with the patient's teeth or cheek wall, making the accurate location of the dispensing feature into the buccal cavity of the patient challenging.

Where highly viscous medicaments are to be administered, a tube of greater diameter will be required than for medicaments having a lower viscosity.

The tube preferably has an average internal diameter of about 0.5 to about 10 mm. In more preferred embodiments the tube has an average internal diameter of about 1 to about 8 mm, about 1.5 to about 5 mm or most preferably about 2 to about 4 mm.

The walls of the tube may have the same thickness as the walls of other parts of the container, or may be different. The tube walls must have sufficient thickness to provide a degree of rigidity to the dispensing tube, such that the distal end of the tube can be easily placed into the buccal cavity of a patient. The average thickness of the tube wall is preferably between about 0.01 and about 3 mm, more preferably between about 0.1 and about 2 mm, and most preferably between about 0.25 mm and about 1 mm.

Thus, the tube preferably has an external diameter of about 0.5 to about 10 mm. In more preferred embodiments the tube has an external diameter of about 1 to about 8 mm, about 1.5 to about 5 mm or most preferably about 2 to about 4 mm.

The tube may be formed of one piece, which is preferably the same piece as the remainder of the container. Alternatively, the tube may be formed of more than one piece. For example, a separate tube attachment may be employed. In such an arrangement, a container designed for use with paediatric patients may have a short tube. It may be useful to lengthen the short tube, for example, to make the container suitable for use with mature patients.

In an alternative arrangement, the container may not include any tube, but simply a tube anchoring feature to which a tube could be coupled.

The medicament which is to be housed in the container is a liquid for buccal administration. In most cases, such medicaments will be viscous and thus, the container must be dimensioned to allow the liquid to be dispensed. A single arrangement of the container of the present invention may not be appropriate for use with every buccal liquid medicament. For example, a medicament which had a high viscosity may require a dispensing tube having a greater diameter than for a medicament having a lower viscosity.

The containers of the present invention may be used to administer any liquid medicament to the buccal cavity of a patient in need thereof. Preferred classes of medicament include but are not restricted to analgesics, anti-convulsants, antidepressants, vasodilators, steroids, opiate antagonists, anaesthetics, antiadrenergic compounds, antiallergic drugs, anti-anginals, anti-asthmatics, antibacterials, anti-coagulants, anti-cholinergics, antiemetics, antiepileptics, antihistaminics, antiinfectives, antiinflammatories, antimigraine drugs, bronchodilators, cardiac depressants, thrombolitics, beta blockers. These medicaments may include controlled substances such as opioids, sedatives, benzodiazepines and stimulants. Specific examples of medicaments which are suitable for use with the containers of the present invention include midazolam, lorazepam, diazepam, paraldehyde, pentobarbital, morphine, carbamazepine, ethosuximide, clorazepate, clonazepam, felbamate, forphenytoin, gabapentin, lacosamide, lamotrigine, levetiracetam, oxacarbacepine, phenobarbital, phenytoin, pregabalin, primidone, tiagabine, topiramate, zonisamide, clobazam, vigabatrin, fentanyl, oxycodone, diamorphine.

As the sealing means of the container is removed, it is envisaged that the container will be gripped between fingers of the user. This may exert pressure on the squeezable region of the container. It is therefore preferred that, in addition to storing the liquid medicament, the sealed container will also contain an amount of air or inert gas (in the event that the medicament is sensitive to air) to ensure that when the seal is removed, the risk of inadvertent discharge of the medicament is reduced.

Certain medicaments which are suitable for use with the containers of the present invention are light sensitive. An example of such a medicament is midazolam. Accordingly, once the containers of the present invention are manufactured, they may be overwrapped with an opaque material.

The container, once appropriately packaged, may be provided as part of a kit. The kit may additionally comprise instructions to administer the medicament into the buccal cavity of a patient. Additionally or alternatively, the kit may comprise additional medicament/s, either packaged in the container's of the present invention or using other types of packaging.

Further preferred features and advantages of the invention are recited in the detailed description and in the claims.

### BRIEF DESCRIPTION OF THE DRAWINGS

An embodiment of the invention will now be described, by way of example, with reference to the accompanying figures.

FIG. 1 shows a perspective view of a container of the present invention.

FIG. 2 shows a perspective view of a ‘card’ of three containers of a first arrangement of the present invention.

FIG. 3 shows a perspective view of a ‘card’ of five containers of a second arrangement of the present invention.

### DETAILED DESCRIPTION OF THE PREFERRED EMBODIMENTS

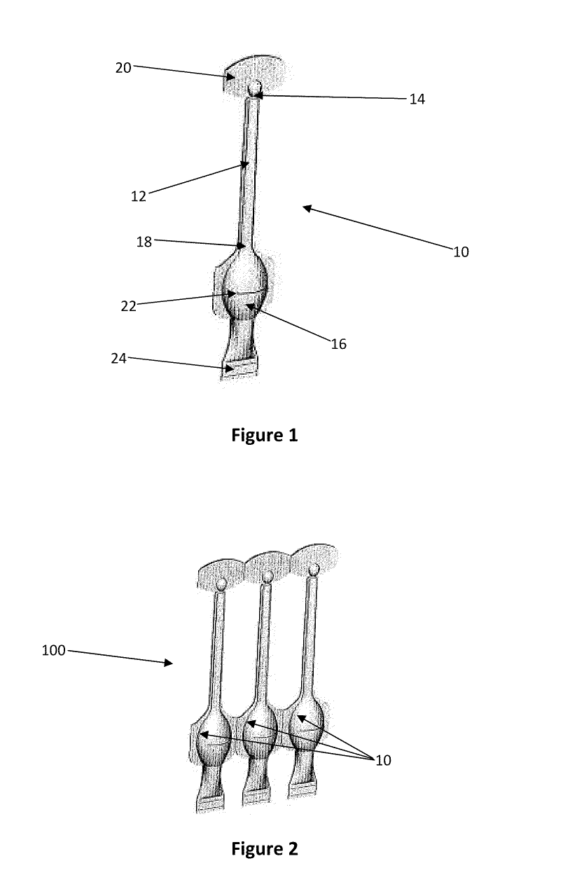

FIG. 1 shows a representation of a container (10) of the present invention. The container (10) includes a dispensing tube (12) having a dispensing feature at the distal end of the tube (12) which is closed by a frangible seal (14). A chamber (16) is provided at the other end of the tube (12). The upper end of the chamber (16) is formed into a transition zone (18), where the tube (12) and the chamber (16) meet. The chamber (16) and tube (12) are in fluid communication.

The tube (12) is of generally circular cross section and is of broadly constant diameter. In the illustrated embodiment, the tube has a length of about 4 cm, which is sufficient to allow the distal end of the tube to be located within the buccal cavity of most patients, without the user's hand having to be located between the jaws of the patient, thus reducing the risk of injury to the patient. In alternative embodiments, longer or shorter tubes may be employed.

At the distal end of the tube (12) a frangible seal (14) is provided. In the illustrated embodiment, this seal takes the form of a bubble in the tab (20) which blocks the tube (12) preventing egress of the medicament, even if the chamber is squeezed. The seal is made frangible by a weakened region of the tube (12), adjacent to the bubble, which enables the seal (14) to be conveniently removed by the user, leaving a smooth, non-jagged edge which is free of burrs.

To further assist the user, a tab (20) is provided. When the dosage contained within the container is to be administered, the user grips the container (10) with the thumb and fingers of one hand and uses the thumb and forefingers of the other hand to grip the tab (20) and provide a rotational force thereto, which tears the tube along the weakened line, resulting in removal of the tab (20) and the seal (14) from the container (10). The tab (20) and seal (14) can then be discarded.

Housed within the container (10) is a dosage of medicament and also a portion of inert gas. The purpose of the gas is to ensure that the pressure exerted on the container (10) by the user when removing the seal does not result in the inadvertent discharge of medicament prior to administration to the patient.

Following removal of the frangible seal (14), the distal end of the tube (12) is placed in the buccal cavity by the user, who grips the container (10) by holding the chamber (16) between a thumb and a finger. Once the distal end of the tube (12) is in position, the user squeezes the chamber (16), forcing the medicament out of the chamber (16), through the tube (12), through the orifice formed by removal of the frangible seal (14) and into the patient's buccal cavity.

The chamber (16) shown in FIG. 1 is lozenge-shaped. The use of chambers shaped in this way is preferable as there are no corners into which medicament can be trapped when the chamber (16) is squeezed to administer the medicament. In the event that a chamber (16) shape is employed which has corners, e.g. a square, rectangle, triangle, etc., any corners should be rounded to prevent medicament becoming trapped.

The chamber (16) is provided with a fill line (22). The purpose of the fill line (22) is to allow the user to verify that the correct volume of medicament is present within the chamber (16) and that there have been no leaks or discharge of the medicament prior to use. In the illustrated arrangement, the user holds the container (10) such that the tube (12) points upward, allowing the liquid medicament to settle at the base of the chamber (16). If the meniscus of the medicament is aligned with the fill line, then a full dose of medicament is present.

At the base of the chamber (16), an identification tab (24) is provided. The purpose of this tab (24) is to bear information relating to the medicament, for example to the name of the drug, the batch number or its expiry date. This information may be printed onto the tab (24) using ink, or embossed on the tab (24).

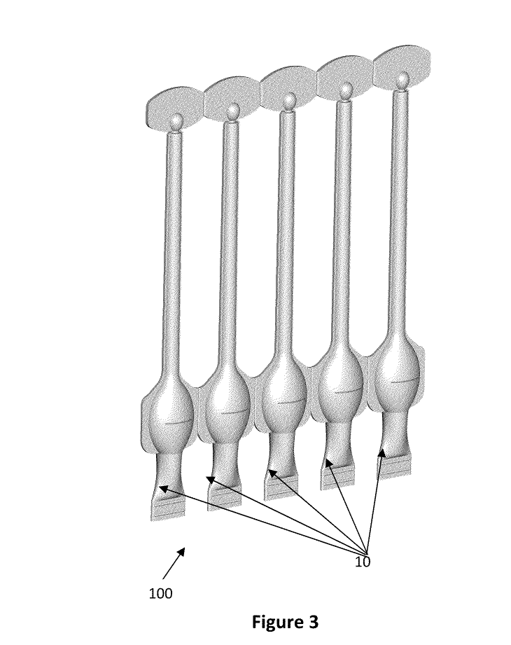

FIG. 2 shows a representation of a card (100) of three containers (10). Containers of the present invention may be prepared using any manufacturing technique known to those skilled in the art. However, injection moulding, especially blow-fill-seal techniques are especially preferred. When such techniques are employed, the containers (10) may be provided in the form of a card (100). In the card (100) illustrated in FIG. 2, three droppers are present. However, cards (100) may be manufactured including any number of droppers. Once the card (100) is manufactured, it may be divided into separate containers (100) and individual containers are required where the medicament packaged therein is for use in treating acute conditions, e.g. seizures or bursts of pain. Where the medicament is used to treat chronic conditions, for example requiring thrice daily administration, it may be preferred to provide the containers (10) in cards (100) of three containers (10)

FIG. 3 shows a representation of a card (100) of five containers (10). The containers (10) illustrated in FIG. 3 differ from those illustrated in FIGS. 1 and 2 in that the tubes have a slightly longer length, about 6 cm.

1. A unit-dose container for administering liquid medicament to the buccal cavity of a patient comprising a chamber, a squeezable region, sealing means, and a dispensing tube in fluid communication with the chamber, the dispensing tube comprising a dispensing feature and having a length of 1 to 12 cm.

2. The container of claim 1 wherein the container comprises liquid medicament housed therein.

3. The container of claim 2 wherein the medicament is a controlled substance.

4. The container of claim 2 wherein the medicament comprises midazolam, morphine, lorazepam, diazepam or diamorphine

5. The container of claim 1, wherein the squeezable region is at least part of the container wall surrounding the chamber.

6. The container of claim 1, wherein the sealing means is a frangible seal.

7. The container of claim 1, wherein the length of the tube is 3 to 7 cm.

8. The container of claim 1, wherein the length of the tube is 4 to 6 cm.

9. The container of claim 1, wherein the container is formed at least partially from polyethylene, polypropylene, polyester or a mixture thereof.

10. The container of claim 1, wherein the container is formed of a single material.

11. The container of claim 1, wherein the container is formed of a single piece.

12. The container of claim 1, wherein the chamber is circular, diamond shaped or lozenge shaped in cross section.

13. The container of claim 1, wherein the container is provided with a fill line.

14. The container of claim 1, wherein the container is provided with an information tab.

15. (canceled)

16. A kit comprising the container of claim 1 and instructions for administration of a medicament to a patient via the buccal cavity using the container.

17. A method for preparing the container of claim 1 in which the container is prepared using injection moulding techniques.

18. The method of claim 16 wherein the container is prepared and filled using blow-fill-seal apparatus.
